(19) United States
(12) Patent Application Publication
(10) Pub. No.: US 2005/0019774 A1
(43) Pub. Date: Jan. 27, 2005

(54) Device for the simultaneous dialysis of a number of fluid samples

(76) Inventors: Anton Horn, Jena (DE); Stefan Kreusch, Golmsdorf (DE); Gunther Sammler, Jena (DE); Renate Bublitz, Jena (DE); Sina Schwedler, Eisenach (DE)

Correspondence Address: TESTA, HURWITZ & THIBEAULT, LLP, HIGH STREET TOWER, 125 HIGH STREET, BOSTON, MA, 02110, US

(21) Appl. No.: 10/498,617

(22) PCT Filed: Dec. 10, 2002
(86) PCT No.: PCT/DE02/04553

(30) Foreign Application Priority Data
Dec. 10, 2001 (DE) 101609752

(51) Int. Cl. C12Q 1/68; C12M 1/34

(52) U.S. Cl. 435/6; 435/287.2

(57) ABSTRACT

The invention elates to a device for the simultaneous dialysis of a number of fluid samples, comprising a vessel for the dialysis fluid, with an inlet and outlet, a fill-level regulation and a sample plate (3) with a number of similar sample vessels (5) of μl range size, arranged in a raster (n×8×12), the upper ends of which are open and the lower ends of which are sealed by a semi-permeable membrane (7) lying in a plane. In the raster region of the sample vessels (5), the sample plate has no regions or elements which extend beyond the plane of the membrane (7), or which form or support gas barriers after dipping in the dialysis fluid (2). The sample plate (3) comprises elements in the boundary region thereof for the escape of air after dipping in the dialysis fluid. Both sample fluids and dialysis fluids are agitated.

### DESCRIPTION

The invention concerns an apparatus for the simultaneous dialysis of a plurality of liquid samples, which are contained in sample wells in a sample plate, and have been brought to this sample plate for a dialysis separation by at lest one semipermeable membrane which is in contact with a dialysate. Such a dialysis system can find general application where, for analytical purposes, specifically a plurality of liquid microsamples is to be investigated regarding their the macro-molecular apportionment and from which microsamples, low molecular weight molecules, which would disturb the analysis, must be separated. The separation is to be carried out in an efficient, easily manipulated manner and at the lowest possible expense. Beyond this, the dialysis system is to be applied advantageously in order to concentrate macromolecular containing microsamples quickly, protectively and without substantial loss. A further area of application is the buffering of samples, especially in the range of the DNA-treatment, for the investigation of proteins and in the case of sequentially occurring enzymatic reactions.

In recent years, highly parallel screening techniques, such as High Throughput Screening=HTS and Ultra High Throughput Screening=UHTS, have been showing up as analytical methods very frequently in analytical work. This has been especially aided by the efforts of the pharmaceutical industry for the capture of targets for the development of newer pharmaceuticals. Also, the proteomanic analysis is vigorously developing itself, and enables a very high sample throughput for the characterization of a multiplicity of proteins of a proteome with biological modifications both in various conditions, such a healthy and ill. Beyond this, the said proteomic analysis is an indispensable aid for many application ranges in biochemical and biotechnical research with high throughput analyses, for example, for the characterization of enzymes in regard to their activity, for the characterization of analytical and preparative chromatographic separations, for the mass centered renaturation of protein samples and for the characterization of nucleic acids.

These high throughput procedures have led, in their general range, to acceptable auxiliary technologies for the development of special analysis procedures. For example, these group themselves around a microtiter of 8×12 analysis positions in the basic area of a well plate of about 12.6×8.6 cm. At the present time, attention is given to a trend for even further compression of the analysis positions on the same basic area of n×8×12, where n can equal 4, or even 16 and further to more densely compacted multiple well plates. Very frequently, the materials of interest can be macromolecular substances such as DNA and its fractionates, proteins, peptides, glycoprotein and synthetic macromolecules as well as combinations of these substances.

Two additional and new procedures, MALDI-MS (Matrix Assisted Laser Desorption Ionization Mass Spectrometry) and ESI-MS (Electrospray Ionization Mass Spectrometry) have been recently developed in the last few years, and have proved themselves as excellent procedures for HTS/UHTS and for the proteomic analysis. In this case, especially in combination with protease digestion methods. In general, a strong tendency toward miniaturization of the methods of analysis must be recognized. Many procedures for the high-throughput screening method show a strong demand for sample preparation with high requirements. Some of the requirements are listed below:
1. the samples may contain only a low concentration of salt or detergent, or the samples must be found in a specific millieu of ionization,
2. the samples, which lie in the μl-volume range, must be treated in a highly parallelized manner, in order to guarantee the required analysis frequency, The treatment must be carried out, uniformly and under standardized conditions for all samples,
3. the recovery for these microsamples in the process of the sample preparation, must be satisfactorily high, and
4. frequently, samples of biological material must be protectively concentrated before the analysis, in order to achieve the necessary desired level of concentration.

For the removal of low-molecular substances, for the transfer of the samples into a defined millieu and for the concentration of macromolecular materials, excellent results have been acquired with the dialysis procedure requiring the use of semi permeable membranes. Consequently, there has arisen a greater number firms offering dialysis procedures, which, for example, can be found collected together under the address of http://biosupplynet.com.

The principal effort of the firms and the inventors for the improvement of the dialysis technology, addresses those problems, which relate to the practical manipulation of the technology. Some of the problems to be overcome are: mixing, recovery of macromolecules, difficulties with the speed of the dialysis process, and operations which are connected with small volumes, i.e., in the μl-range.

Where the MALDI and the ESI-MS procedures are concerned, the present state of the technology finds the removal of salts, detergents and other contaminating substances necessary.

Possibilities, which could improve the quality of the essential mixing of a dialysis solution, are described in U.S. Pat. No. 5,183,564 and in U.S. Pat. No. 6,176,609. U.S. Pat. No. 6,176,609 in particular describes a general procedure for mixing in a plurality of vessels. Giving consideration to the universal trend for miniaturization, a series of solutions were suggested. Also, U.S. Pat. No. 6,039,871 teaches of equipment which can be looked at as being disposable and the dialysis of 10 to 100 μl samples is foreseen as being in special vessels which can be encapsulated, one in the other. In U.S. Pat. No. 5,733,442 a closed microdialysis system is proposed, which is marked by microchambers, which can be screwed together, and possess two dialysis membranes and a special stirring device.

The difficulties which arise in connection with recovery of small analyte volumes is given attention by the inclusion of a special capture chamber in U.S. Pat. No. 6,217,772.

In the documents U.S. Pat. No. 5,783,075 and U.S. Pat. No. 5,503,741 a proposal for floating dialysis in vessels of a special configuration is made known. Where the floating dialysis is concerned, there are also offerings from the firm PGC, the firm Daigger and the firm Pierce, where the widely publicized slide dialysis system is treated.

In order to increase the rapidity of the dialysis, giving consideration to the ESI-MS technology, a capillary dialysis system is taught by U.S. Pat. No. 5,954,959.

Where a multiplicity of samples must be treated, proposals are put forth in U.S. Pat. No. 4,450,076. In these proposals the dialysis vessel is placed about a central axle, and is turned by means of rotation. Offers from PGC Scientifics Corp. bring forward a central axis oriented, equilibrium chamber, which is sealed by Teflon coated screws. Pierce Biotechnology, Inc. offers a microdialysis system for 12×20−100 μl volumes.

The proposed solutions of the problem, however, are not adaptable to the demands of higher throughputs, such as are necessary for HTS, UHTS and for proteomic analysis because of the following, because they:
1. are not acceptable for highly parallel microplate technology especially where corresponding liquid treatment is necessary,
2. a simultaneous and essentially uniform dialysis executed for all samples in a greater sample count in the μl range is prohibited,
3. a satisfactorily sufficient recovery of these small volumes fro secondary analytical procedures is not allowable,
4. a correspondingly high dialysis speed is not provided,
5. the volumes required for the dialysate are inadequately large, or
6. because of their complicated manner of application, the said solutions are not practical in routine operation with a high throughput.

Thus, the invention has the purpose, of dialysing simultaneously a multiplicity of micro samples in the μl-range essentially uniformly, wherein the manipulation is easily carried out and at as low a cost as possible, whereby these dialyses can be executed quickly and, if required, in an automatized manner, within the requirements of the modem screen and analysis methods.

In the case of a large throughput of samples, the dialysis permits a sufficiently high recovery of small volumes for secondary analytical procedures.

In accord with the purpose of the present invention, there is proposed an apparatus for the simultaneous dialysis of a plurality of liquid samples, wherein:
there is included a dialysis vessel with a dialysate as well as means for the inflow and outflow of the dialysate, which remain in connection with a level control,
there is included at least one sample plate, which is either immersed in, or is in contact with, the surface of the dialysate, as well as being held by holding elements, in or on the surface of the dialysate, with a matrix (n×8×12) arrangement of a plurality of equal sample wells, which arrangement is acceptable and known for liquid handling technique in microplate technology and said wells have holding capacities for microliter volumes, whereby, of the sample wells, respectively, the upper ends are open and the lower ends are closed by a semipermeable membrane lying in a plane, and the sample plate in the area of the said matrix of the matrix of the sample wells has no gas barrier forming or supporting zones or elements which extend beyond the plane of the semipermeable membrane and the said sample plate 3 has, in its rim areas, elements, such as openings for the release of air imprisoned by touching contact with the dialysate,
and besides the above, the said apparatus contains means for the movement of the plate and the dialysate.

Enabled by the acceptability of the sample plate used in the dialysis system, for the said liquid handling technique for the microplate technology of liquid, samples can be prepared with a high degree of throughput in accord with this technology, then subsequently be employed for their purpose and finally recovered for further use. With the special design of the sample plate, those air barriers which obstruct the dialysis upon the immersion of, or the displacement of the plate into or onto the dialysate surface are avoided. Should procedurally evolved gases migrate into the contact zone between the dialysate and the sample plate, then these can be forced out by deaerating apparatuses, so that in any case, an unbroken contact can be assured between the dialysate and the sample plate without the said gas barriers, such as air bubbles and the like. This disturbance-free contact is an essential presupposition for simultaneous and essentially uniform dialysis for each of the large number of samples in the μl range. This effect is essentially supported by the movement of the sample liquids and the dialysate, so that, predominately, no secondary membrane formation between the dialysate and the sample volumes can form and the dialysis can continue with unbroken continuity. To serve this purpose, the dialysis vessel possesses at least one entry and one exit opening, in order that the materials which are accumulated in the dialysate can again be continuously expelled from the said dialysate and continually a dialysate with equal dialysis acceptance power remains available in the system. The entry and exit flows are, meanwhile, connected by a level control, in order to hold constant the conditions of dialysis on the semipermeable contact between the sample liquid and the dialysate, thus maintaining the above advantages with consideration for level control.

By means of the movement of not only the dialysate, but also of the sample liquid, this movement being done by a known shaking device, with which the sample plate is connected, not only is the high dialysis effectivity itself attained, but also special usages, notably dialysis-effectivity is achieved. Of the latter, a dialysis of detergents which form micellla, is already enabled.

With the above stated features, the realization of variously designed dialysis systems is enabled, wherein the said dialysis systems can carry out different applications, either manually or with automatic drive, these being independent therefrom, as to whether the sample plate, for instance, by means of a pivot or a shaking arm lies on the dialysis vessel or floats in the dialysate which is present in the container.

In the most simple case, without limiting the invention, the sample plate may consist of a plate with cylindrical recesses or wells in the receiving means for microliter volumes. On the underside of the sample plate are the borings (sample containing recesses) either respectively closed by a common or by individual dialysis membranes, which, for example, are adhesively held on the underside of the plate. They may also be welded, bonded, or sprayed on. The dialysis membranes can also consist of more than one layer.

A cover or an adhesive film of a releasable closure of the upper end of the sample vessel protects the sample material which is in the sample wells, and blocks any evaporation and contamination of the small quantity of sample in the microliter range.

Described and explained in the subordinate claims, are a multitude of advantageous embodiments of the invented features. In this way, the dialysis vessel with the accepted sample plate becomes an integral part of a circulation system. In such a circulation system, for example, with a pump controlled, recycling apparatus, it is possible that an ion-exchange device or a detergent capturing adsorber could be placed. Such an addition would hold the concentration of the substances which are to be removed from the dialysate to a very small level, and thereby the speed of the dialysis procedure would be increased and the necessary volumes of the dialysate would be simultaneously minimized. Also the use of bound substances which form complex substances is possible, in order to remove metal ions.

In the following, the invention is described and explained with the aid of embodiments as shown in the drawing. There is shown in:

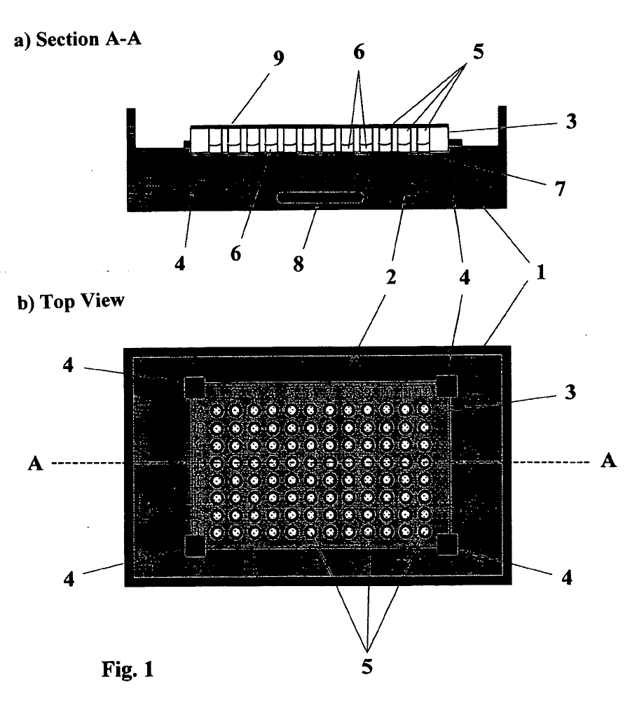

FIG. 1: A sample plate, secured at the base of a dialysis vessel by feet,

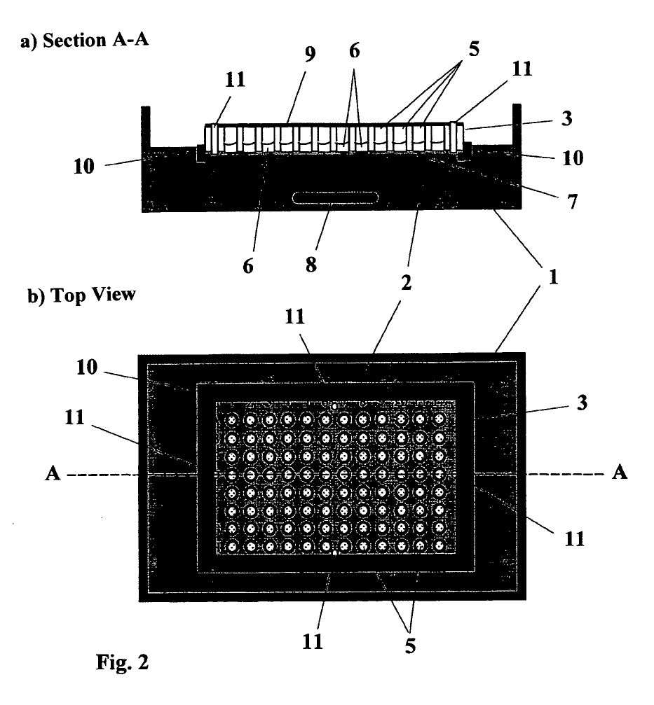

FIG. 2: An apparatus with a floating holder of the sample plate on the surface of the dialysate,

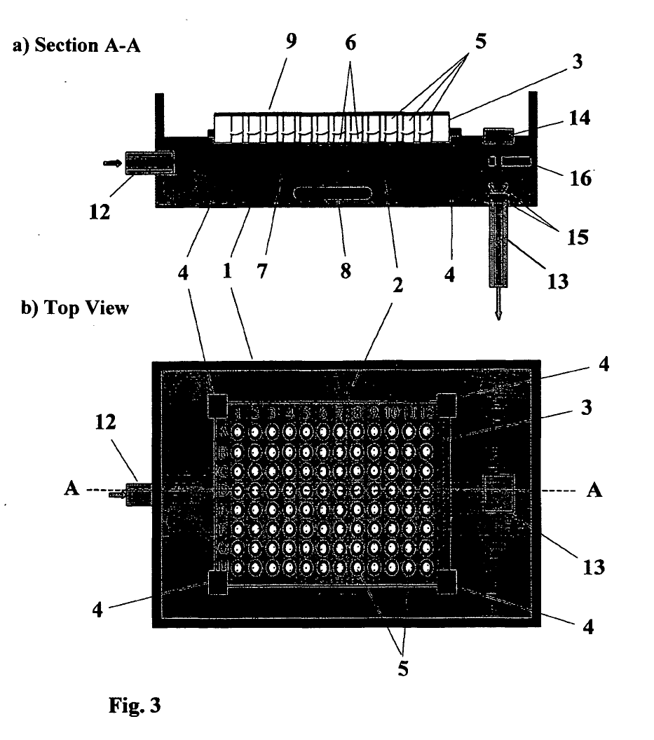

FIG. 3: A dialysis vessel with the holder of the sample plate in accord with FIG. 1, with both entry and exit fittings for the dialysate.

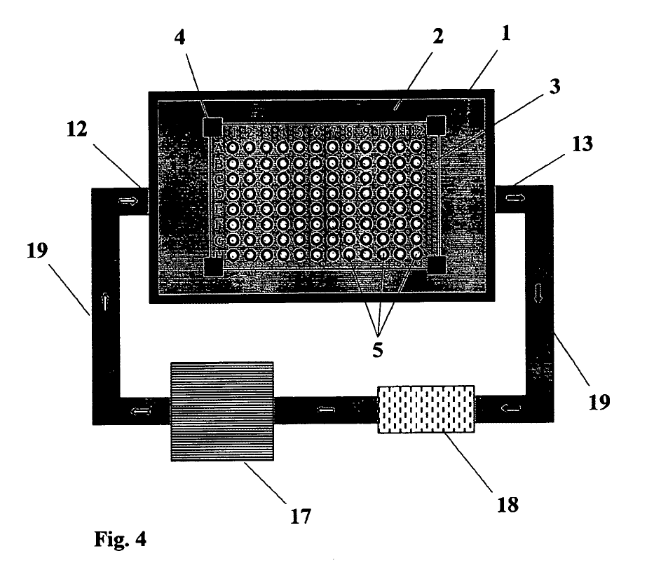

FIG. 4: A dialysis vessel with a sample plate in a circulation system for the removal of interfering substances from the dialysate,

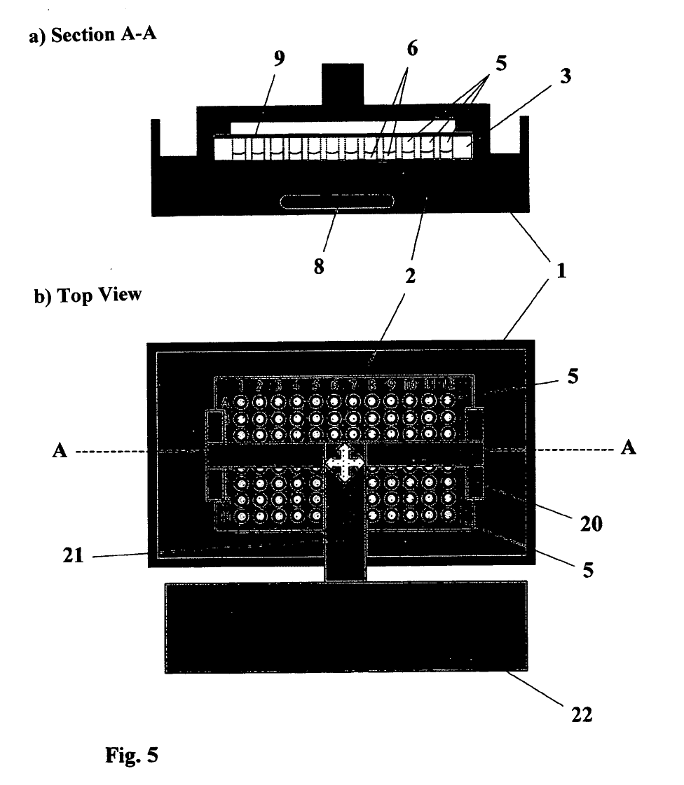

FIG. 5: An apparatus for dialysis, wherein the sample plate is held in a shaking device to create turbulence in said dialysis apparatus,

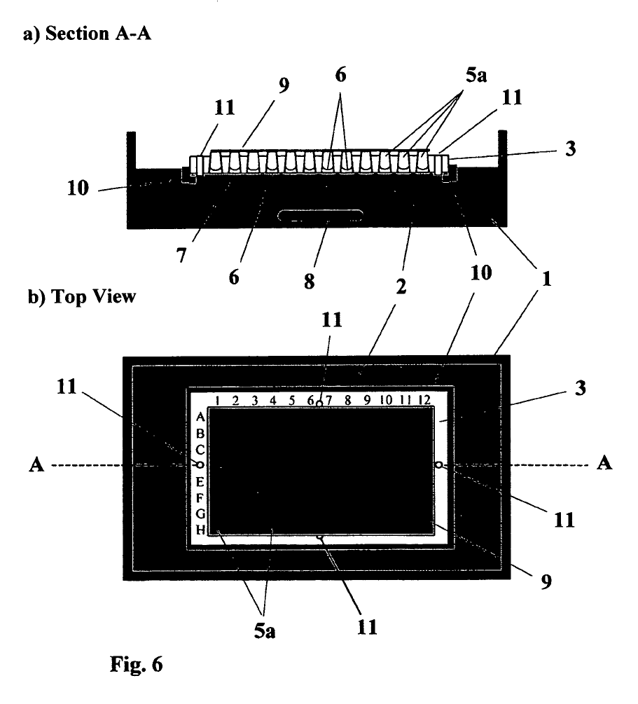

FIG. 6: A floating holding means for the sample plate (see FIG. 2) with conical sample wells,

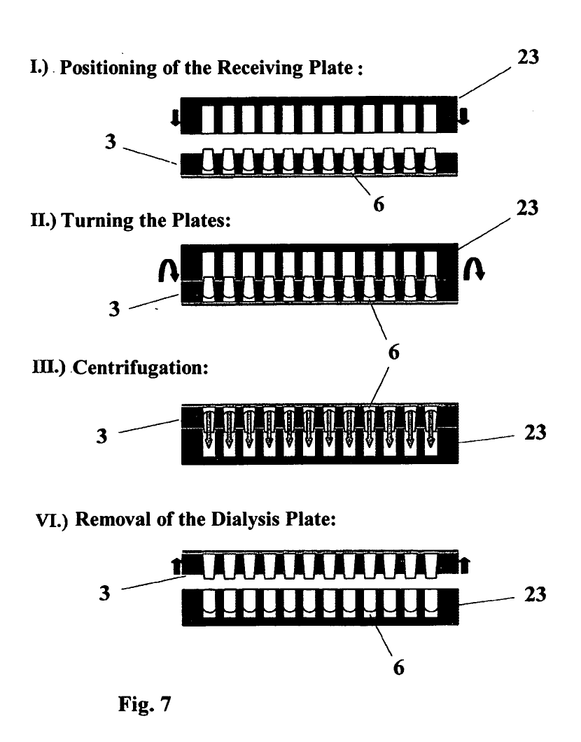

FIG. 7: A sequential run of a procedure to transfer dialyzed sample material subjected to centrifugation out of the sample plate with conical sample wells into a receiving plate with cylindrical wells,

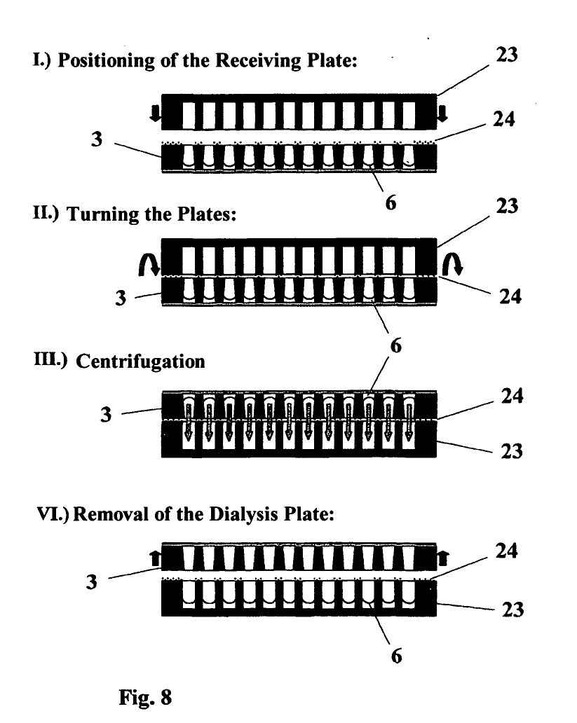

FIG. 8: A second sequential run of a procedure similar to FIG. 7, showing a sealing means between the sample plate and the receiving plate, and

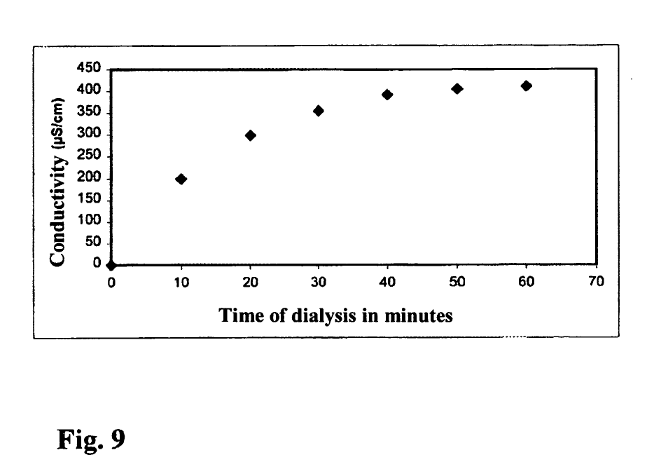

FIG. 9: A graph showing the conductivity of the dialysate during a period of dialysis.

In FIG. 1 is shown a dialysis container 1 with a dialysate 2 therein. On the bottom of, and within the dialysis container, is to be found a sample plate 3, which is secured by a holder 4. This sample plate 3 consists of a plate shaped, basic body in which, and within the specifications of a known liquid handling technique for acceptable microplate technology, is placed an 8×12 matrix of aligned cylindrical wells 5 for the acceptance of sample material 6, the content of each well being in the microliter range.

On the underside of the sample late 3 is found a dialysis membrane 7, this membrane being semipermeable, common to all sample wells and secured on the rims thereof by adhesive. By means of this dialysis membrane 7, each individual portion of the sample material 6, which is within the wells 5, stands respectively in contact with the dialysate 2. For this purpose, the sample plate 3 is so supported by the holder 4, that the said plate 4 is immersed, with its dialysis membrane 7, into the dialysate. By means of the dialysis membrane 7, the exchange of small molecules is possible, in accord with the exclusion threshold, since a concentration equilibrium between the dialysate 2 and the liquids of the sample material 6 is in force. The removal of the said small molecules out of the material 6 of the samples is accomplished by the effort of the said solutions to establish the mentioned equilibrium between the two compartments. Large molecules are restrained from passing through the dialysis membrane 7.

The plane of the dialysis membrane 7 incorporates, in a way, also the lowest level of the sample plate 3, during its operation in accord with its application. There exists in this matrix area of the sample wells 5 no zones or elements for stabilization fastening, manipulation or the like, or even yet areas dependent upon fabrication, which would protrude from the sample plate 3 outward and beyond, which would contactingly impinge on the dialysate 2, or be immersed therein. Further the arrangement is such that no extension of the said zones or elements exist, which would interfere with the uniformly running dialysis process by introducing air or creating air barriers.

By means of a known magnetic stirrer 8 on the bottom of the dialysis container 1, the dialysate is held in motion, in order that a concentration gradient on the dialysis membrane 7 be held as small as possible and also to accelerate the dialysis. An adhesive foil 9, serving as a releasable closure of the upper rims of the sample wells 5, protects the sample material 6 which is found therein and prevents an evaporation or a contamination of the sample very small volumes.

FIG. 2 depicts a construction, which is very similar to that of FIG. 1. The difference, in this case, is that the sample plate 3 is not supported by foot or structural holding elements 4 rigidly connected to the bottom of the dialysis container 1, but is held by means of a framelike, float element 10 directly on the surface of the dialysate. This is accomplished in such a manner, that the dialysis membrane 7 lies on this surface. This mode of holding is independent of the level of the dialysate 2 in the dialysis container 1.

Additionally, the sample plate 3 has air escape openings 11, which allow gas collecting under the sample plate 3 to bleed out, thereby assuring an unbroken contact of the dialysis membrane 7 with the surface of the dialysate 2. This complete contact coverage forms the necessary preparation for a simultaneous and essentially uniformly completed dialysis for each sample of this large number of samples in the μl-volume range.

Because the sample plate 3, at least in the matrix area of the sample wells 5, possesses no elements (for holding or the like), which would protrude downward beneath the plane of the dialysis membrane 7 and thus immerse themselves in the dialysate 2 or cause turbulence in the same, either of which would disturb the desired uniformity of simultaneous analyses, very quickly essential characteristics for value-determining usage of the sample plate 3 in a dialysis system were immediately taken advantage of, in order to avoid air locks, or at least not to support them, in the contact zone of the dialysate 2 against the sample plate 3. If, nevertheless, gases evolved from processing appeared in this zone, then, these gases, as mentioned above, could disperse through the air escape openings 11 and emerge above trough the sample plate 3. Instead of the air escape openings 11, other gas dispersing elements were given consideration, such as edge phase-changing, or the like. To enhance clarity, details of the air escape openings 11 (or other deaeration equipment) were not explicitly shown in each figure presentation.

FIG. 3 shows an apparatus for dialysis, wherein the sample plate 3 (as in FIG. 1) is supported in the interior of the dialysis container 1 on the floor thereof by means of feet or standard holding devices 4. The dialysis container 1 possesses in this case, a feed fitting 12 as well as a outlet fitting 13 for the dialysate 2. The level of the dialysate 2 is regulated by means of an adjustable float 14 with a float actuated valve 15. In this case, the float 14 is guided to be vertically movable in a float track 16. The advantage of this, is to be found in the continuous content balancing of the dialysate 2. In this way, the adjustment of a concentration equilibrium between the dialysate 2 and the respective sample material 6 contained in the wells 5 can be avoided, also the speed of the dialysis is greater and the removal of low molecular substances from the sample material 6 is fundamentally improved. Naturally, the float valve system (12-17) can be replaced by an electronic level controller which regulates the feed at inlet 12.

FIG. 4 shows an apparatus for dialysis, wherein the dialysis container 1, which is shown in top view, demonstrates the therein placed sample plate 3 (see FIGS. 1 and 3), and shows the inlet 12 as well as the outlet 13 as being components of the circulation system for the through-flow of the dialysate 2. The exchange of the dialysate 2 not carried out, as in the apparatus of FIG. 3, through an open system, but rather by the dialysate 2 being transported by a pump 17, through a filter cartridge 18 and into a line 19, with the circulation system being completed by passage through the dialysis container 1. The direction of the dialysate 2 is indicated by an arrow, whereby the black arrows symbolize the exit flow of the dialysate 2. Upon its exit out of the dialysis container 1, the dialysate 2, for example, can be enriched with ions and/or detergents from the sample material 6. By means of one or more sorbents in the filter cartridge 18, these components can be removed from circulation. The cleaned dialysate 2 thus migrates, as the white arrows show, back into the dialysis container 1. Advantageously, here, the ubiquitous applicability of the dialysate 2 contributes to:
(a) the said avoidance of the adjustment of a concentration equilibrium, between the dialysate 2 and the sample material 6 held respectively in the sample wells 5 and
(b) the thereto connected advantages for dialysis (see the embodiment example of FIG. 3).

FIG. 5 depicts an apparatus for dialysis (once again sectional profile and top views), wherein the sample plate 3 is neither anchored to the bottom of the dialysis container 1 (see FIG. 1) nor is it floating on the dialysate 2 surface in the said dialysis container 1 (see FIG. 2). Rather the sample plate 3 is held by a holder 20, which is also connected to a shaking device 22 through a shaker arm 21. The shaker 22 serves, as the white, crossed arrows indicate, for the horizontal movement of the sample plate 3 along the surface of the dialysate 2 and also moves the sample plate 3 within the amplitude and frequency limits as directed for a shaker installed for microtiter sample plates as these limits are defined for laboratory operation. In this way, the sample material 6 found in the sample wells 5 of the sample plate 3 is thoroughly mixed, which acts against the establishment of concentration gradients in the said sample material 6, as well as in the dialysate 2, which the shaker 8 also affects. This has the favorable advantage, that the somewhat hindering construction of secondary membranes, which are necessary for many dialysis processes, may be omitted. Also, the transporting away of gas bubbles in the area of the membrane is favored by this shaking motion.

The advantage of this apparatus is, that not only is the recirculation and cleaning associated with the content balance of the dialysate 2, as is described for the embodiments of FIG. 3, 4, omitted, but also the speed and completeness of the dialysis is improved. To preserve clarity, a combined presentation with the said, and previously described embodiments, is not specifically illustrated in the attached drawings. In an additional embodiment example, not shown here, the motion of the samples and dialysate can be carried out with the same positive effects also by being coupled with an ultrasonic mixer.

FIG. 6 shows (likewise in sectional profile and top views) an apparatus for dialysis, wherein the sample plate 3, as is the case in FIG. 2, floats on the surface of the dialysate 2. The sample plate 3 does not possess, as was the case in the previously described embodiments, sample wells with cylindrically parallel walls, but sample wells 5a which are conically tapered, in the form of a cone frustum with respectively larger lower openings, which are closed by the dialysis membrane 7. The said wells 5a have, in comparison to the lower openings, smaller upper openings. The smaller upper openings create an advantageous shape-closure (see FIG. 7) for the transfer of the sample material 6 after the dialysis (following the dialysis) into the individual well volumes, in the same matrix in another sample receiver plate. This procedure is in accord with known microtiter plate technology. Easily recognizable in the top view presentation of FIG. 6 is adhesive foil 9 over the matrix arrangement of the sample wells 5a, which foil prevents evaporation, spilling, and contamination of the sample material 6, which is in the wells 5a during the dialysis or can occur even during transport.

FIGS. 7, 8 show, schematically, respectively in sectional views through the plate, a sequential run of the procedure through centrifugation, out of the sample plate 3 with conical sample wells (see FIG. 6) into a receiver plate 23 with cylindrical wells. The receiving plate 23, in an upset position, is placed on top of the sample plate 3, whereupon the two are turned over in common. By means of centrifugation, the sample material 6, which is originally in the sample plate 3 is transferred to the receiver plate 23. Subsequently, the sample plate 3 and the receiver plate 23 are taken apart. As a centrifugal device, the known laboratory centrifuge for microtiter plates can be used. In FIG. 7, the upper openings of the wells 5 are smaller than the openings of the sample container of the receiver plate 23 which confronts them. On this account, the already described satisfactory shape-fit assures the penetrative interconnection as shown in the drawing 7. Conversely, in FIG. 8, we see the confronting well openings of the sample plate 3 and the receiving plate 23 respectively equal in size. In this case, a required tighter shape fit is assured by means of an intermediately inserted sealing means 24 between the sample plate 3 and the sample plate 23.

In the following four embodiments is shown, how, with the described apparatuses, different substances can be dialyzed.

### APPLICATION EXAMPLE NO. 1

Low-molecular substances in 96 samples such as p-nitrophenol (p-NP) and sodium chloride, are to be uniformly removed, in short dialysis periods, wherein the reception of the sample plate 3 in a shaking device (see FIG. 5) is provided and which said plate 3, in accord with FIG. 4, is connected into a circulating system for the dialysate 2.

For this situation, into the 8×12 dialysis wells, which are closed with a VSMP Millipore membrane (0.025 μm), each 100 μl of a 1.5 mM p-NP solution in 50-mM diethanol-amine buffer pH=9.8 (DEA), which, in addition, contains 750 mM NaCl, is pipetted and dialyzed against a volume, which is only 11 times greater than a volume of 110 ml deionized water for 2 hours. The dialysate 2 is circulated by a hose pump. In the circuit is integrated a deionizing column (Eco Pac, 10 ml, Bio Rad) (see FIG. 4). During the dialysis, the sample plate 3 is continually shaken, which sample plate is closed with adhesive foil 9 and is fastened in the holder 20 of the shaping apparatus (see FIG. 5). The effectivity of the separation of the lower molecular nitrophenols following the dialysis is checked with the absorbencies of the outlet solution. The absorbency measurement of the outlet solution, which emerges from the dialysis, is carried out with a DEA-buffer solution, pH=9.8 in a microtiter plate and the absorbencies read off on a display. For the absorbency measurement of the 96 dialyzed samples, 3 aliquots are taken by pipette from the sample plate 3, with 50 mM DEA-buffer solution pH=9.8, mixed in a microtiter plate and measured in a display.

The comparison of the absorbency in the 96 positions of the dialysis module is shown in Table 1. The measured absorbencies have been reduced by the blind buffer value.

TABLE 1
Comparison of the Absorbencies at
405 nm (A₄₀₅) before and after Dialysis

| | A₄₀₅ Analysis | Dilution | | |
|---|---|---|---|---|
| | Solution | Factor | | |
| | Av. Val. | Analysis | | |
| Samples | (n = 96) | Solution | A₄₀₅ Total | p-NP(%) |
| p-NP pre Dialysis | 0.397 ± 0.005 | 30 | 11.910 | 100 |
| p-NP post Dialysis | 0.028 ± 0.002 | 3.75 | 0.105 | 0.88 |

The values indicate, that under the described conditions, more than 99% of the p-NP can be removed. The distribution of the absorbency values after the dialysis show, that the dialysis speed in all 96 dialysis wells is very much the same. Besides the comparison of the absorbencies before and after the dialysis, the conductivities of the employed samples were measured and were compared with the conductivities after the dialysis of the 96 dialysis wells. From these values a residual capability of conductivity was determined, in relation to the outlet solution of 0.2%, which, in any case, confirms the effectivity of the dialysis.

### APPLICATION EXAMPLE NO. 2

For the removal of lower molecular ions from proteins, in 8×11 positions of the sample plate 3, per well, 75 μl of concentrated solution of an alkaline phosphatase (14.5 μg/ml) was added to IM DEA buffer pH=9.8, and dialyzed for one hour against a large volume of 470 ml deionized water. The sample plate 3, which is placed as a floating element 10 on a polysterol framing, and floats on the dialysate 2, which is kept in motion by the magnetic stirrer 8 (see FIG. 2). The continuous removal of the low molecular substances in the sample wells 5 is monitored by the measurement of the conductivity (see FIG. 9) in the said dialysate 2.

After 60 minutes of dialyzation, 60% of the ions which can be dialyzed have been removed. The determination of the enzymatic activities of the alkaline phosphatase in the 88 occupied dialyzation wells of the sample plate 3 and a comparison of the enzymatic activity with the outlet solution yielded a recovery of the enzymatic activity of 90.4+4.3%.

### APPLICATION EXAMPLE NO. 3

On the example of Triton X-100 (TX-100), the point is to determine, if it is possible to remove this much used detergent by dialysis from analysis samples. To this end the sample plate 3 is, respective by wells, charged with 7511 of a 0.5% aqueous solution of Triton X-100 and dialyzed for 8 hours vs tap water. The sample plate 3 was closed by adhesive foil 9, and again placed in the holder 20 of the shaker apparatus (see FIG. 5) for continuous agitation. During this part of the operation, the same was immersed in 170 ml of tap water as a dialysate 2, which was renewed at flow rate of 170 Ml/min in a circulation system in accord with FIG. 4.

In order to capture eventual volume changes, the sample plate 3 was weighed before and after the dialysis. For the determination of the effectivity of the removal of the Triton by dialysis, aliquots were taken by multipipettes from the dialysis containers of the module, mixed in a microtiter plate with 30% n-propanol and measured in a fluorescence display at an excitation wave length of 270 nm from an emission wave length of 310 nm. The Triton X-100 which was subjected to dialyzation, after dilution in 30% n-propanol was measured under the same conditions in a microtiter plate. For the correction of the measured values and for the regulation of the linearity of the range of measurement, both the propanol solution in 56 positions of the microtiter plate (blind values) and three standard concentrations of Triton X-100, between 12.5 and 50 μM under the same conditions were measured. The results are summarized in Table 2. The measured average values were reduced by the determined propanol blind value:

| | | Dilution | | |
|---|---|---|---|---|
| | Fluorescent | Factor | | |
| | Analysis Solution | Analysis | Fluorescence | |
| Samples | Av. Val. (n = 96) | Solution | Total | TX-100 (%) |
| TX-100 | 739.6 ± 11.83 | 201 | 148 660 | 100 |
| pre Dialysis | | | | |
| TX-100 | 62.39 ± 6.24 | 30 | 1 872 | 1.26 |
| post Dialysis | | | | |

The measured fluorescences following the dialysis in the 96 dialysis wells of the sample plate 3 show, that under the described conditions, 98.7% of the Triton X-100 was uniformly removed from all 96 of the dialysis wells.

### APPLICATION EXAMPLE NO. 4

The sample plate 3 can also be employed for the simultaneous concentration of 96 samples. For this purpose, in accord with each well, 100 μl of a 0.3% Dextra-blueing solution with a multi-pipette was placed in the sample wells 5 of the sample plate 3. The sample plate 3 was subsequently fixed on the floating frames 10 (see FIG. 2) of polysterol, which were laid on 100 ml of a 30% aqueous polyethylene glycol solution (PEG 40 000). After 45 minutes, the volume reduction was quantitatively determined. To this, from 88 out of the 96 positions was taken, per position, 30 μl with a multi-pipette and transferred into a microtiter plate, which, per container, contained 120 μl 50 mM DEA-buffer solution. In the remaining 8 positions of the microtiter plate, instead of the sample solution, each was given 30 μl reference solution (0.3% Dextra-blueing solution). The microtiter plate was then, after intensive mixing, measured in a display at 620 nm. A comparison of the determined absorbency of samples and reference solution shows, under the described conditions, the measured absorbency.

TABLE 3
Comparison of the Absorbency of Dextra Blueing before and after
concentration

| Samples | A₆₂₀ |
|---|---|
| Reference Solution | 0.291 ± 0.012 (n = 8) |
| Samples after Concentration | 0.465 ± 0.020 (n = 88) |

The absorbencies have increased by a factor of 1.6. That means, that after 45 minutes the volume of the samples is reduced by 37.5 μl, and indeed relatively uniformly, as may be seen by the distribution.

### APPLICATION EXAMPLE NO. 5

For the dialysis of plasmid DNA, in a 96 well dialysis-plate, 110 μl of the samples having plasmid DNA pc DNA3.1hcSΔE44-N63(6.5 kb) was treated against buffer Tris 10 mM pH 8 and subjected to shaking and mixing. To the samples, paranitrophenol (PNP) was added in the end concentration of 978 μM and the reduction of the concentration was measured respectively after two and four hours.

The content of DNA in the samples was likewise determined after two and four hours in, respectively, eight parallel samples of a concentration with the aid of optical density. The detection was carried out at 400 nm for PNP and 260 nm for the plasmid DNA.

The table below shows the balance of the plasmid-DNA: That is, the concentrations were computed with a standard series of plasmids by optical density.

Table:

| | After 4-hour |
|---|---|
| Output DNA | Dialyzation, DNA |
| in μg/ml | in μg/ml |
| 5 | 4.55 |
| 10 | 9.32 |
| 50 | 50.79 |

The balance of PNP in the samples, as per a standard series for the samples where n=32:

| | | PNP after 2 hour | PNP after 4 hour |
|---|---|---|---|
| Item | Output PNP | Dialysis | Dialysis |
| Concentration in the | 978.6 | 38.96 | 0.56 |
| sample in μM | | | |
| Standard deviation | 1.01 | 4.6 | 0.60 |
| in μM | | | |
| % from output | 100 | 3.9 | 0.06 |

Reference Numbers and Corresponding Components
1 Dialysis container
2 Dialysate
3 Sample plate
4 Holder
5 Well, (5a conical tapered well)
6 Material of the sample
7 Dialysis membrane
8 Magnetic stirrer
9 Adhesive foil
10 Float element
11 Air escape opening
12 Entry fitting (feed)
13 Outlet fitting (exit)
14 Float
15 Float valve
16 Float guide
17 Pump
18 Filter cartridge
19 Line, for circulation of liquid
20 Holder (FIG. 5)
21 Shaker arm
22 Shaker
23 Receiving plate (receives contents of 3)
24 Sealing means

1-35. (Cancelled)

36. An apparatus for simultaneous dialysis of a multiplicity of liquid samples, the apparatus comprising:
a dialysis container adapted for receiving a dialysate therein, means for inflow and outflow of the dialysate, and a level controller;
at least one sample plate disposable in the container for contact with the dialysate, the sample plate including a multiplicity of equal sample wells with a (n×8×12) matrix arrangement, wherein the wells define holding capacities for microliter volumes, open upper ends, and lower ends closed by a semipermeable dialysis membrane lying in a plane, whereby the sample plate does not protrude beyond the plane of the semipermeable membrane in an area defined by the matrix arrangement, the sample plate further including means for releasing air imprisoned between the sample plate and the dialysate;
means for holding the sample plate in contact with a surface of the dialysate; and
means for moving at least one of the sample plate and the dialysate.

37. The apparatus of claim 36, wherein the sample wells are substantially cylindrical.

38. The apparatus of claim 36, wherein the sample plate comprises a geometry of conventional microtiter plates.

39. The apparatus of claim 36, wherein at least some of the lower ends of the sample wells are respectively closed by single dialysis membranes.

40. The apparatus of claim 36, wherein at least some of the lower ends of the sample wells are closed by at least one common dialysis membrane.

41. The apparatus of claim 36, wherein the dialysis membrane comprises a plurality of membrane layers.

42. The apparatus of claim 36, wherein the dialysis membrane is sprayed upon the sample plate.

43. The apparatus of claim 36, wherein the dialysis membrane is adhesively connected to an underside of the sample plate.

44. The apparatus of claim 36, wherein the dialysis membrane is at least one of bonded, welded, and sprayed on an underside of the sample plate.

45. The apparatus of claim 36, wherein an adhesive foil is provided as a releasable closure of the upper ends of the sample wells.

46. The apparatus of claim 36, wherein a cover is provided as a releasable closure of the upper ends of the sample wells.

47. The apparatus of claim 36, wherein the dialysis container comprises at least one inlet and at least one outlet, at least one of which is connected to a float valve.

48. The apparatus of claim 47, further comprising means for removal of substances to be dialyzed out of the dialysate, the removal means in fluidic communication with the at least one outlet and the at least one inlet.

49. The apparatus of claim 48, wherein the removal means comprises at least one ion exchanger.

50. The apparatus of claim 48, wherein the removal means comprises an absorber for binding detergents.

51. The apparatus of claim 48, wherein the removal means comprises chelate forming substances.

52. The apparatus of claim 47, wherein dialysate flow through the at least one inlet and through the at least one outlet is adjustable.

53. The apparatus of claim 52, wherein the dialysate flow is adjustable by at least one of a valve and a positioning element.

54. The apparatus of claim 47, wherein the dialysis container, including the at least one inlet and the at least one outlet, forms at least a portion of a circulation system for the dialysate, the circulation system further comprising a rotary pump.

55. The apparatus of claim 36, wherein the sample plate is held in the dialysis container by at least one stand element disposed on a bottom of the dialysis container.

56. The apparatus of claim 36, wherein the sample plate is held by at least one or more float element disposed in the dialysate contained in the dialysis container.

57. The apparatus of claim 36, wherein the means for holding the sample plate includes means for providing movement and mixing of a sample material contained in at least one of the sample wells.

58. The apparatus of claim 57, wherein the means for providing movement and mixing comprises a shaker.

59. The apparatus of claim 57, wherein the means for providing movement and mixing comprises an ultrasonic source.

60. The apparatus of claim 36 further comprising at least one monitoring element for at least one of measuring and treating the dialysate.

61. The apparatus of claim 60, wherein the at least one monitoring element comprises means for simultaneous measurement of conductivity.

62. The apparatus of claim 60, wherein the at least one monitoring element comprises means for simultaneous measurement of optical density.

63. The apparatus of claim 60, wherein the at least one monitoring element comprises a fluorescence detector.

64. The apparatus of claim 60, wherein the at least one monitoring element comprises a thermometer.

65. The apparatus of claim 60, wherein the at least one monitoring element is provided for control of the dialysis process.

66. The apparatus of claim 36 further comprising means for transferring a sample from the sample plate.

67. The apparatus of claim 66, wherein the means for transferring the sample comprises a receiving plate, which can be inverted and placed on the sample plate, the receiving plate including a plurality of sample wells with a (n×8×12) matrix arrangement corresponding to the matrix arrangement of the sample wells of the sample plate.

68. The apparatus of claim 67, wherein the means for transferring the sample comprises using centrifugation.

69. The apparatus of claim 67 wherein the sample wells of the receiving plate define openings that correspondingly engage openings defined by the sample wells of the sample plate to establish a shape fit.

70. The apparatus of claim 69, wherein the sample wells of the sample plate comprise a conical shape and the openings of the sample wells of the sample plate are individually smaller than corresponding openings of the sample wells of the receiving plate, where the openings coincide when the receiving plate is set upon the sample plate.

71. The apparatus of claim 69, wherein the openings of the sample wells of the sample plate are individually substantially equal in size to the openings of the sample wells of the receiving plate.

72. The apparatus of claim 67, wherein the means for transferring a sample further comprises a seal between the sample plate and the receiving plate, the seal selected from at least one of a sealant and a paste.

73. The apparatus of claim 36, wherein the means for the moving the dialysate comprises a magnetic stirrer.
